(19) United States
(12) Patent Application Publication
(10) Pub. No.: US 2026/0137359 A1
(43) Pub. Date: May 21, 2026

(54) SMART ALIGNMENT FOR DIGITAL RADIOGRAPHY

(71) Applicant: CARESTREAM HEALTH, INC., Rochester, NY (US)

(72) Inventors: Huihong DUAN, Shanghai (CN); Wayne GONG, Shanghai (CN); Clifford CHEN, Shanghai (CN); Xiaohui WANG, Pittsford, NY (US); Ken SUN, Shanghai (CN)

(21) Appl. No.: 19/258,979

(22) Filed: Jul. 3, 2025

Related U.S. Application Data
(60) Provisional application No. 63/668,328, filed on Jul. 8, 2024.
(60) Provisional application No. 63/668,330, filed on Jul. 8, 2024.
(60) Provisional application No. 63/668,332, filed on Jul. 8, 2024.
(60) Provisional application No. 63/668,333, filed on Jul. 8, 2024.

(51) Int. Cl. A61B 6/46; A61B 6/00; A61B 6/42; A61B 6/58

(52) U.S. Cl. CPC A61B 6/463; A61B 6/4233; A61B 6/4405; A61B 6/4464; A61B 6/4476; A61B 6/582; A61B 6/587

(57) ABSTRACT

An x-ray imaging system includes an x-ray source assembly having a transmitter and a video camera. A free standing digital radiographic (DR) detector includes a receiver, wherein the transmitter and receiver are configured to determine an spatial orientation of the digital radiographic detector relative to the x-ray source assembly. A digital display presents a live video image of a patient with a current position of the DR detector overlayed on the video image of the patient. A DR detector having built-in AEC sensors may display the current position of each of the AEC sensors in the overlay relative to the DR detector position based on the determined orientation of the DR detector. Each of the AEC sensors are individually activatable, and an AEC selection interface is configured to receive an operator selection of one or more of the AEC sensors to be activated.

### CROSS REFERENCE TO RELATED APPLICATIONS

This application claims priority to: U.S. patent application Ser. No. 63/668,328, filed Jul. 8, 2024, in the name of Duan et al., and entitled SMART ALIGNMENT FOR DIGITAL RADIOGRAPHY; U.S. patent application Ser. No. 63/668,330, filed Jul. 8, 2024, in the name of Duan et al., and entitled AUTOMATIC ALIGNMENT FOR DIGITAL RADIOGRAPHY; U.S. patent application Ser. No. 63/668,332, filed Jul. 8, 2024, in the name of Duan et al., and entitled AUTOMATIC EXPOSURE CONTROL FOR DIGITAL RADIOGRAPHY; and U.S. patent application Ser. No. 63/668,333, filed Jul. 8, 2024, in the name of Duan et al., and entitled IMAGE INFORMATION FOR DIGITAL RADIOGRAPHY, which are all hereby incorporated by reference herein in their entirety.

This application is related in certain respects to: U.S. Pat. No. 8,873,712, issued Oct. 28, 2014 to Wang et al. ; U.S. Pat. No. 9,179,886, issued Nov. 10, 2015 to Stagnitto et al. ; U.S. Pat. No. 10,285,656, issued May 14, 2019 to Wang et al. ; U.S. Pat. App. Pub. No. US 2024/0197270 A1, published Jun. 20, 2024, to Wang et al. ; and Int'l. App. No. PCT/US 23/71473, published Feb. 6, 2025, to Sun et al., all of which are hereby incorporated by reference in their entirety as if fully set forth verbatim herein.

### BACKGROUND OF THE INVENTION

The subject matter disclosed herein relates to alignment in digital radiography (DR) imaging. In particular, proper alignment between the radiation source and the x-ray detector is one of the embodiments for generating radiographic images.

Typically, for a fixed DR system, alignment between the radiation source and the x-ray detector is generally ensured by a complex and sophisticated mechanical device. The x-ray detector inserted in a holder can be well aligned with the radiation source, after the mechanical device is properly installed and calibrated. In which case, it is usually called as an in-holder or in-bucky exposure case. An out-of-bucky case represents that the detector will be removed from the holder and manually positioned on the exam table or hospital bed. In such cases the patient's body will likely partially or completely obstruct the detector. The alignment between the detector and radiation source will be disrupted, since this alignment can no longer be achieved through the calibrated mechanical device. The aforementioned issues will result in technicians having to rely solely on visual cues to align the radiation source and detector, which is time-consuming, laborious, and makes it difficult to achieve accurate alignment. As a result, the quality of the captured images may be compromised, leading to either poor image quality or a higher rate of retakes.

Previous systems for aligning the radiation source and x-ray detector have been based on detecting the temporal differences between a set of signal emitters and signal receivers. The signal emitters installed on the radiation source, or tube head or x-ray source assembly, will initially emit two sets of signals sequentially, which are then received by the signal receivers installed on the detector. The relative position and angle between the radiation source and the detector can be calculated and established based on the actual distance and angle between the emitter and receiver. These parameters are calculated by a control processor based on the temporal differences of the signals they receive and their known geometric installation location. These systems are simple, effective, and easy to implement. One disadvantage of these previous solutions, which utilize signal emitters and receivers to locate the radiation source and the x-ray detector, are the limited capability to provide location information. This limitation is based on the number and installation position of the emitters and receivers.

Another disadvantage of the previous solution, which relied on calculating the temporal differences between emitters and receivers, is that they must be precisely installed to establish an ideal geometric relationship between them. This ensures that the calculations based on their ideal geometric location and the temporal differences are valid and reliable. Another disadvantage of the previous solution is that the entire system is specifically designed for the DR detector being installed in a bucky. It subsequently led to the difficulty in supporting the free standing out-of-bucky DR detector situations, where this alignment system is most needed to support the patient positioning, since the calculation model will become ineffective when the detector is removed from its holder or bucky.

The discussion above is merely provided for general background information and is not intended to be used as an aid in determining the scope of the claimed subject matter.

### BRIEF DESCRIPTION OF THE INVENTION

An x-ray imaging system includes an x-ray source assembly having a transmitter and a video camera. A free standing digital radiographic (DR) detector includes a receiver, wherein the transmitter and receiver are configured to determine a spatial orientation of the digital radiographic detector relative to the x-ray source assembly. A digital display presents a live video image of a patient with a current position of the DR detector overlayed on the video image of the patient. A DR detector having built-in AEC sensors may display the current position of each of the AEC sensors in the overlay relative to the DR detector position based on the determined orientation of the DR detector. Each of the AEC sensors are individually activatable, and an AEC selection interface is configured to receive an operator selection of one or more of the AEC sensors to be activated.

In one embodiment, an imaging system for radiographically imaging a patient includes a manually movable x-ray source assembly having a transmitter and a video camera. A free standing manually positionable DR detector includes a receiver. The transmitter and receiver are configured to communicate with each other in order to determine a spatial position of the x-ray source relative to the digital radiographic detector. A display screen coupled to the video camera displays a live video image of the patient and the system is configured to display an outline of a current position of the DR detector overlayed on the video image of the patient while the x-ray source is being manually moved.

In one embodiment, a method of operating an x-ray imaging system includes positioning a DR detector adjacent to a patient and positioning a radiation source such that the patient is located between the DR detector and the radiation source. Guidance information is displayed on a digital display screen and is used for positioning the radiation source in a properly aligned imaging position, whereafter capturing a radiographic image of the patient may be undertaken.

In another embodiment, an x-ray imaging system for capturing radiographic images of a patient includes an x-ray source including a transmitter. A free standing manually positionable DR detector includes a plurality of AEC sensors and a receiver. The transmitter and receiver are configured to determine an orientation of the digital radiographic detector relative to the x-ray source. Each of the plurality of AEC sensors are individually activatable using an AEC selection interface configured to receive an operator selection of one or more of the plurality of AEC sensors to be activated. The x-ray imaging system is configured to activate the operator selected ones of the AEC sensors for radiographic imaging of the patient. The AEC selection interface indicates a current position of each of the plurality of AEC sensors relative to the detector based on the determined orientation of the digital radiographic detector.

Hence, a well-designed smart alignment system that offers both visual guidance information and automatic alignment between the radiation source and the detector for out-of-bucky scenarios will not only significantly expedite the positioning process and enhance image quality, but also decrease the retake rate, leading to time and workload savings.

The present invention discloses an embodiment to locate and track the position between the radiation source and the x-ray detector. Subsequently, guidance information is provided to assist in positioning the x-ray source and the DR detector. Furthermore, the system can be configured to automatically align the x-ray source and the DR detector to ensure optimal alignment, thereby affording high-quality images and reducing the need for image retakes. These techniques can solve most of the problems associated with previous solutions, which were limited in their ability to apply to only in-bucky cases with limited support and guidance.

The present disclosure introduces an alignment system to overcome the problem and disadvantages of the previous solutions and methods. This system consists of a visualization module, a positioning module, a calibration module, an automatic alignment module and a data processing module 462. The location information of the radiation source and the x-ray detector is initially provided by the positioning module, which is then fused with a video stream captured from the visualization module through the data processing module 462. The fused information will be displayed on the digital display 464 in real-time, so that it can provide vivid guidance during the system positioning process.

The system may also be configured to automatically align the x-ray radiation source and the DR detector. This is achieved after establishing a transformation relation between the source and the detector through the positioning module, which is then passed to the automatic alignment module. Consequently, the radiation source and DR detector are adjusted to an appropriate position accordingly.

In one embodiment, a system assembly includes a visualization module, a positioning module, a calibration module, an automatic alignment module and a data processing module 462. With the positioning module, this embodiment enables the radiation source to track the location of the x-ray detector. Then a coordinate transformation relation between the radiation source and the detector is established, which will be passed to the calibration module, the visualization module, and an automatic alignment module for further use.

The summary descriptions above are not meant to describe individual separate embodiments whose elements are not interchangeable. In fact, many of the elements described as related to a particular embodiment can be used together with, and possibly interchanged with, elements of other described embodiments. Many changes and modifications may be made within the scope of the present invention without departing from the spirit thereof, and the invention includes all such modifications.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings below are intended to be drawn neither to any precise scale with respect to relative size, angular relationship, relative position, or timing relationship, nor to any combinational relationship with respect to interchangeability, substitution, or representation of a required implementation., emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIGS. 1A-1B are diagrams of a DR detector and AEC sensors; and

FIG. 2 is a parameter table;

FIG. 3 is a flow chart for capturing radiographic patient images;

FIG. 4 is a schematic diagram of a radiographic imaging system;

FIG. 5 is a schematic diagram of an x-ray source assembly and a DR detector;

FIG. 6 is a schematic diagram of a radiographic imaging system arranged near a patient; and

FIG. 7 is a digital display presenting live video with a semi-transparent overlay showing guidance information.

### DETAILED DESCRIPTION OF THE INVENTION

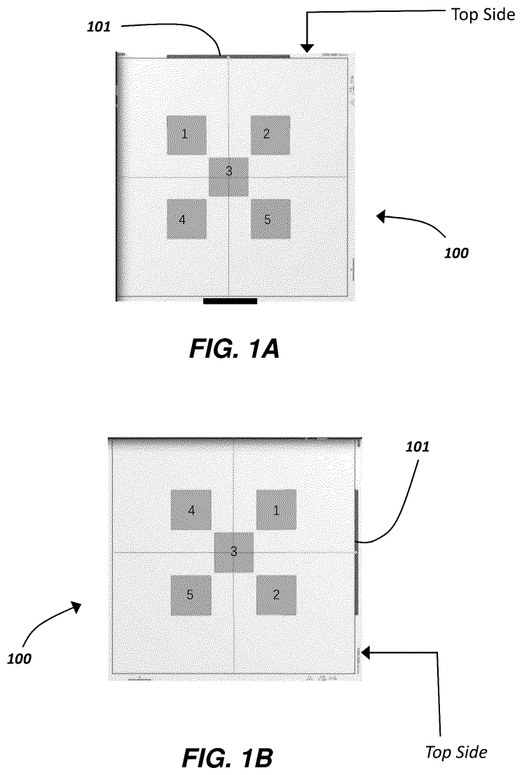

With reference to FIG. 1A and FIG. 1B, there is illustrated a digital radiographic detector 100 which includes, in this example, five built-in AEC sensors designated with numerals 1 through 5, however, DR detectors may be configured with three or less AEC sensors. The detector 100 may include one or more transmitters and/or receivers 101 (Tx/Rx) along one side, or border, two sides or all sides, of the DR detector 100 sufficient to determine an orientation of the DR detector 100. Such an orientation may have, for example, a 90°resolution, or some other amount of angular resolution, such that one side of the detector, such as a side designated as the “top” side, may be determined to be facing generally upward, downward, or to the left or right. Based on any of these potential orientations of the DR detector 100 during x-ray imaging of a patient, it is important for an operator to know the positions and designations of the AEC sensors 1-5 relative to a reference side of the DR detector 100. In the example of FIGS. 1A and 1B, the reference side is illustrated as the top side. As the DR detector 100 may be positioned at various angles when placed next to a patient to be imaged, an operator of the imaging system will have to select which one or more of the AEC sensors 1-5 are to be activated for radiographic imaging based on their location. An operator selection interface may be provided on a digital display, as described herein, for an operator to select which one or more of AEC sensors 1-5 are to be activated during radiographic imaging. The operator interface may indicate the currently determined positions of the AEC sensors 1-5, relative to a top side of the DR detector 100, based on sensing data obtained from Tx/Rx 101. The orientation of the DR detector 100 as shown in FIG. 1A may be considered a 0° orientation, while the orientation of the DR detector 100 as shown in FIG. 1B may be considered a 90° orientation as it is rotated clockwise 90° compared to the orientation illustrated in FIG. 1A. Thus, an operator selection interface presented to an operator may display an arrangement of AEC sensors in a 1-2-3-4-5 fashion (0° orientation), as shown in FIG. 1A, or in a 4-1-3-5-2 fashion (90° orientation) as shown in FIG. 1B. Using this exemplary system and method, the operator will be able to select for activation the AEC sensors as required for patient imaging.

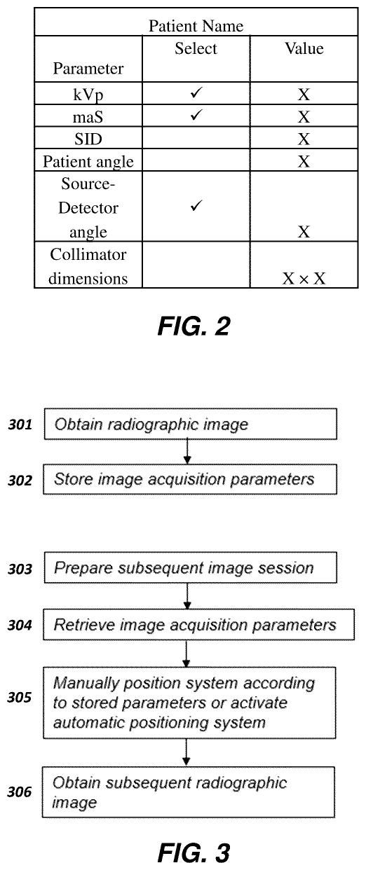

The exemplary x-ray imaging systems described herein may include electronically stored imaging parameters that may be accessed and displayed by an operator. FIG. 2 illustrates an exemplary table of recorded imaging parameters for a patient which, in this example, is stored according to patient name and may be retrieved from electronic memory for display by an operator of the x-ray imaging system. Imaging parameters may be stored according to other designations such as an imaging exam type, e.g., Chest PA, for example. Parameter data is identified in a first column, as shown in FIG. 2, which parameter data may vary for each patient, depending on type of parameter data that has been obtained, or was previously selected to be stored, for later use. An operator may select, via the second column as shown in FIG. 2, e.g., using an operator interface, which one or more of the available parameters may be applied to a present imaging exam for the patient. In the example of FIG. 2, the operator has selected energy levels kVp and mAs, as well as the source-detector angle. The recorded parameter values (X) are shown in column 3 of FIG. 2 and may be reused or adjusted by the operator for a current patient exam. In addition to kVp and mAs (x-ray energy), source image receptor distance, patient body angle, such as in a patient bed, the source-to-detector angle, and collimator (aperture) dimensions, to control an exposure area on the patient, may be recorded for later use, as well as many other alternative imaging parameter data.

FIG. 3 illustrates a flow chart of the presently disclosed method whereby a patient is radiographically imaged 301 and the acquisition parameter data for that imaging session is stored for later use 302. In preparing for a subsequent imaging session 303, the patient's name may be entered into an imaging system data base for retrieving the stored parameter data 304. The retrieved parameter data may be used by an operator to manually set-up and reposition the system 305 in the same manner as was recorded, or the operator may activate an automatic function to reposition and reset the imaging system according to the stored parameter data. A subsequent image of the named patient may then be acquired using the stored parameters 306.

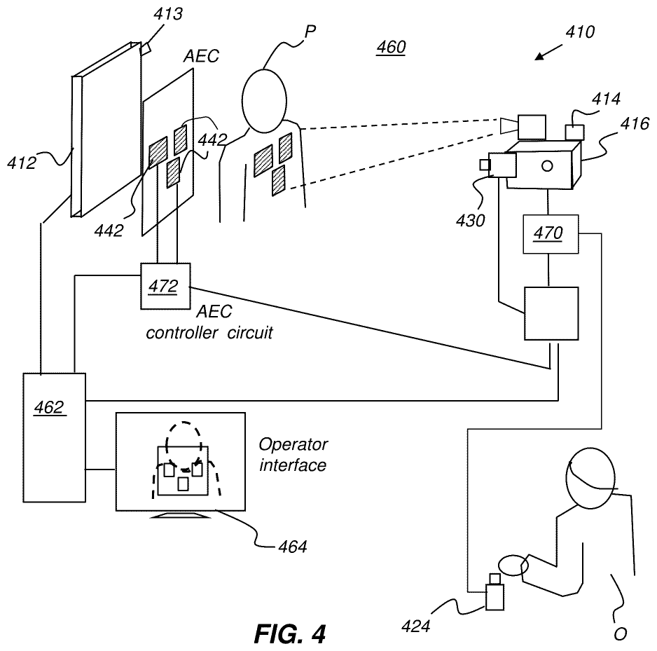

FIG. 4 shows a schematic diagram of an exemplary radiographic imaging system 460 and a visualization module 410 that may be used in determining and visually indicating a position of the DR detector 412 and AECs 442 relative to a patient P. The DR detector 412 may be positioned in a bucky attached to a wall or to a tower and may be vertically moveable therein, manually or under motorized control. The DR detector 412 may be an unconnected free standing detector that is manually positioned behind a patient P. Similarly, the x-ray source assembly 416 may be manually moveable by operator O or may be moved under motorized control. One or more AECs 442 may be selected or enabled for use by an operator O via an operator interface displayed on digital display 464 and input device 424 for a current imaging session of patient P. A reference position of the patient P on digital display 464 serves as a basis for determining the relative spatial coordinates of the AECs that may be available and used for subsequent display. Consistent with one embodiment of the present invention, the reference position relates to part of the patient P that is to be imaged, so that positioning of the AEC sensor elements is determined relative to the position of the DR detector 412 and the outline of the patient P shown on digital display 464.

Visualization module 410 may include a number of components that are used to determine the spatial location of the DR detector 412 relative to x-ray source assembly 416. In the embodiment of FIG. 4, video camera 430 and its associated control logic circuit 470 are part of visualization module 410, used to provide positioning information to data processing module 462 which obtains and displays an image of patient P on digital display 464 together with the relative location of DR detector 412 and AEC sensors 442. Other arrangements of sensors, emitters, and receivers can be used as part of visualization module 410 for obtaining related reference position data.

The reference position of patient P can be detected using emitted signals of a transmitter 414, as part of a positioning module, as described herein, such as an electromagnetic radio-frequency (RF) signal, an ultra wide-band signal, or on three dimensional accelerometer data emitted from emitters positioned on a bucky or other holder, or from positions on a detector, on an AEC sensor, or on the patient P. The signal can alternately be emitted from a transmitter 414 that is coupled to the x-ray source assembly 416, with the signal reflected back, or detected by receivers 413 on the DR detector 412, as described herein, which form a complementary portion of the positioning module.

Light sources and reflective elements for alignment of the x-ray signal to the DR detector; combined with tools such as triangularization, can also be used as part of visualization module 410 to identify a reference position, in conjunction with video camera 430, using methods known to those skilled in the position-sensing arts. FIG. 4 shows a transmitter 414 for emitting a signal and a receiver 413 that can be used for this purpose. The reference positions may be fixed according to the spatial arrangement of components installed as part of x-ray system 460.

In one embodiment, for example, the position of the DR detector 412 is the reference position. The position of patient P is then determined and, accordingly, used for relating the positions of one or more AEC elements 442 to the reference position. In another embodiment, an outline of the DR detector 412 provides the reference position and additional information from known AEC sensor positioning is used to relate the positions of AEC sensors 442 to the DR detector 412. A display step using visualization module 410 is executed for displaying the identified position of the radiation energy sensor elements on digital display 464 relative to the reference position of the DR detector 412.

As shown in FIG. 4, the position of AEC sensors 442 can be displayed on digital display 464. This display can show an image or an outline of the patient P or other subject, or may show the outline of other reference locations on the AEC apparatus or on imaging detector 412. It can be appreciated that there are a number of ways for obtaining positional coordinate data in an imaging apparatus, including methods that use light, RF signals, ultrasound, or other signal types. It is noted that the apparatus and methods of the present invention can be used with any type of AEC controller circuit 472, including the conventional sensor element arrangement of FIG. 4 and the configurable arrangement of sensor elements 1-5 shown in FIGS. 1A-1B.

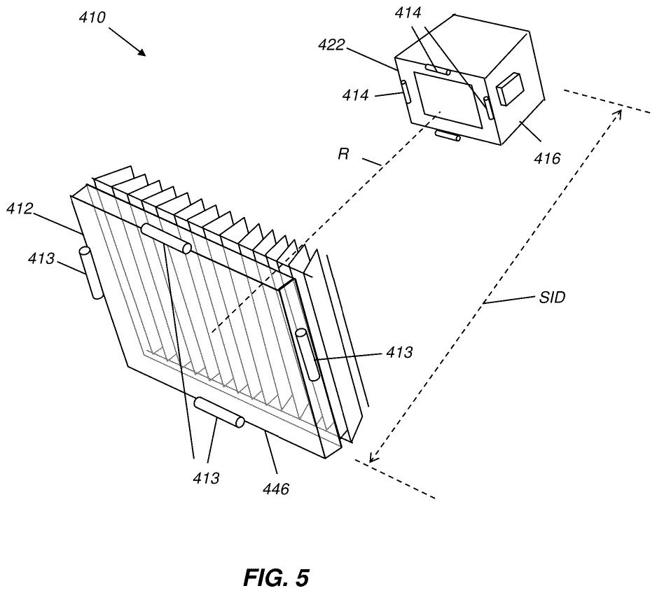

The perspective view of FIG. 5 shows the use of a positioning module 410 that is energizable to sense the relative spatial relationship between x-ray source assembly 416 having a radiation path represented as path R and DR detector 412 sensitive to radiant energy and positioned adjacent the patient for forming the radiographic image of the patient and to generate one or more output signals indicative of the relative spatial relationship, including alignment and distance SID. Receivers 413 are disposed outside the imaging area of the DR detector 412 and may receive an electromagnetic field having time-varying vector directions or signal that is generated by transmitters 414 shown coupled to the x-ray source assembly 416, such as mounted near collimator 422.

Bucky 446 may hold DR detector 412. In an alternate embodiment, the signal transmitter/receiver configuration is reversed: signals are generated from one or more emitters 413, detected by receivers 414 coupled to x-ray source assembly 416. The transmitter/receiver each may include an inclinometer, accelerometer, compass, gyroscopic, or other device for obtaining or calculating an angular measurement can be provided on either or both receiver or transmitter. The position-sensing signals from positioning module 410 go to a data processing module or system 462 that provides calibration and combines video data from the video camera for presentation on a digital display as shown in FIG. 4.

In the alternative embodiment, one or more transmitters 413 on the DR detector 412 may transmit signals detectable by sensors 414 attached to the x-ray source assembly 416. The DR detector 412 may have attached thereto, or embedded therein, one or more transmitters 413 such as electromagnetic coils that generate an electromagnetic field or signal that is detected by one or more sensors 414, shown mounted on the x-ray source assembly 416. In one embodiment, transmitters 413 may include inclinometers for detecting a positional orientation and transmit positional orientation data to data processing module 462 which may be processed for determining relative spatial position.

It can be appreciated by those skilled in the position-sensing arts that there are a number of possible configurations that can be used as a positioning module 410 for position sensing and for providing data for angle, source-to-image-distance (SID) data, data for tracing the DR detector 412 outline, and centering data, when the DR detector 412 is positioned behind or underneath a patient or other object. Centering data relates to the position of the center of DR detector 412 which may be used to generate a cursor overlayed on a display screen 464 that indicates the center of the DR detector 412. The centering data may also be used in combination with other known data related to the size, orientation, and shape of the DR detector 412 and its AEC sensors 442 to generate cursors of various shapes, such as rectangular, to be overlayed on a video image using display screen 464. Source-to-image distance (SID), here the distance between the x-ray source assembly 416 and the DR detector 412 may also be determined.

The transmitters/receivers 413, 414, may transmit analog signals or one or more data values, for example. Position signals can be sent from any of a number of transmitters, including inclinometers, radio-frequency devices, electromagnetic coils, and audio or ultrasonic signals, for example. Transmitters and sensors may be located at edges of their respective devices or may be integrated therewithin. In one embodiment, a processor may be utilized in each of the DR detector 412 or x-ray source assembly 416 to process received positioning signals.

It can be appreciated that any number of possible arrangements of transmitters and sensors may be used in an arrangement similar to that shown in FIG. 5 for determining parameters such as angular orientation, aim centering, source-to-image distance (SID), and other variables that are of interest for obtaining a suitable radiographic image, whether for a mobile, hand held, or fixed-position radiography system. It can be appreciated by those skilled in the position-sensing arts that there are a number of possible configurations that can be used with transmitter/sensor combinations for position sensing and for providing data for angle, SID, data for tracing the DR detector 412 outline, and centering information where DR detector is positioned behind, or underneath a patient. The positional relationship as between the DR detector 412 and the x-ray source assembly 416 may be indicated on the display screen 464.

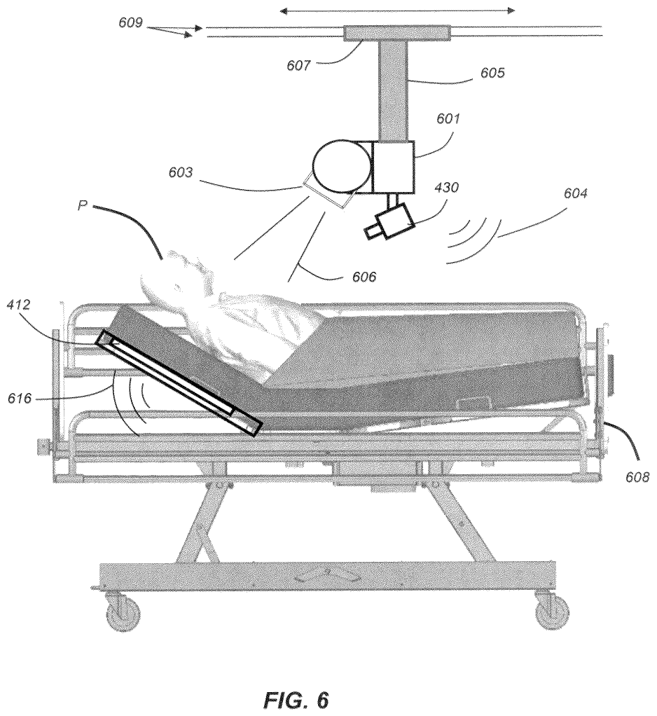

FIG. 6 illustrates an exemplary radiographic imaging system that may be deployed in medical imaging facilities. A movable tube head 601 includes an x-ray source, and has a collimator 603 attached thereto, which tube head 601 may be mounted on an overhead tube crane that includes an extendable vertical support column 605, to which the tube head 601 is attached, and a movable crane base 607, to which the extendable vertical support column 605 is attached. The movable crane base 607 is attached to crane tracks 609 which are affixed to a ceiling of the patient room. When the crane base 607 is moved along the crane tracks 609, such as by a remote controllable motor drive, the tube head 601 may be moved to a desired position. The tube head 601 may also be manually moved by grasping the tube head 601 and pushing or pulling the tube head 601 along tracks 609. The collimator 603 may include an electronically controlled collimator 603 having four individually movable blades for controlling a size of a rectangular aperture which, in turn, controls dimensions of an x-ray beam emitted by the x-ray source. The crane base 607 may also be be attached to second transverse tracks (not shown) to allow remote controlled movement of the tube head 601 along a transverse direction. Typically, the overhead tube crane movements may be configured to be perpendicular to each other and both parallel to a ceiling of the room containing the radiographic imaging system. The extendable vertical support column 605 may also be configured to be telescopically extendable and retractable vertically. The crane base 607 includes an electric motor for controllably driving the crane base 607 along the tracks 609. Movement of the overhead tube crane allows controlled positioning of the tube head 601 in relation to the patient bed 608 and the DR detector 412 located therein. After controllably positioning the tube head 601 in relation to DR detector 412, for example, the x-ray source therewithin may be remotely and controllably fired to emit x-ray beam 606 to expose a patient lying on the patient bed 608 over the DR detector 412.

An operator control system as illustrated in FIG. 4 herein may include a data processing system or module 462 for controlling operation of the radiographic imaging system 460 described herein. The processing module 462 may include a wired coupling or a wireless transmission capability for communicating with and controlling movement and operation of the overhead tube crane, the digital detector 412, a digital video camera 430, as well as the tube head 601 and the x-ray source(s) therein, such as a power level and/or firing sequence of the x-ray source(s), and timing of exposures to be captured by DR detector 412. The tube head 601 and digital camera 430, which may comprise a video camera capable of capturing live video and still images and may include a wireless communication capability 604, and digital detector 412 may also include wireless communication capability 616, for exchanging data and receiving commands and instructions from data processing module 462. The data processing module 462 may comprise a PC or other computing system and includes connected I/O devices such as a keyboard/mouse and digital display 464 for operator O use. The processing system 462 may transmit captured radiographic images to the medical imaging facility, may synchronize an image capture sequence of the DR detector 412 with firing of the x-ray source assembly 416 in tube head 601.

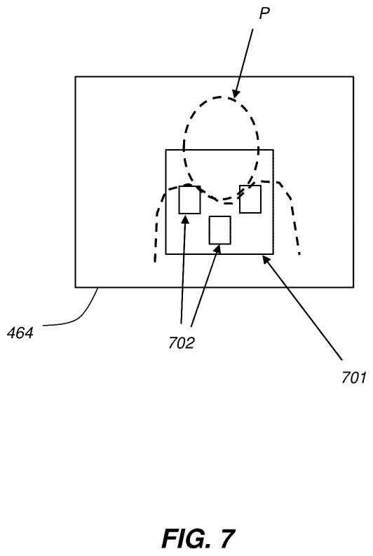

The processing module 462 may be used by operator O to obtain radiographic images of patient P without requiring operator O to have a direct line of sight of the patient P, rather, the operator O may rely on a live video image of the patient P using the digital display 464. The data processing module 462 may be located in a control room of a medical facility on a different floor from the patient room, or even in a different building of the medical facility. As shown in FIG. 7, to properly position the tube head 601 and the detector 412, the operator O may make use of the live video digital image on digital display 464 as captured by camera 430 attached to tube head 601 and aimed at patient P. The video camera 430 may capture and transmit a live video image of the patient P for display to an operator O on digital display 464 (see, e.g., FIG. 4). Based on a calculation of the current position of detector 412, data processing module 462 may be configured to overlay a movable semi-transparent cursor 701, resembling and representing a rectangular shape of the detector 412, on the digital display 464 to indicate an actual live current position of the detector 412 to an operator O. Based on the displayed cursor 701 position, the operator O may further selectively manually adjust the position of the DR detector 412 underneath the patient P until the DR detector 412 is in a satisfactory position for initiating a radiographic image capture of patient P as indicated by the cursor 701 which adjusts together with movement of the tube head 601 and the live video image. Thus, the position of the DR detector 412 as illustrated by the rectangular cursor 701 overlaying the live video image of the patient P may be used by the operator O to verify accurate DR detector 412 placement. The overlayed cursor 701 may include sections 702 indicating positions of the AEC sensors 442. Alternatively, the operator O may manually move the tube head 601 into a proper alignment. Alternatively, an automatic alignment module, as part of the data processing system 462, may automatically move the tube head 601 into an aligned position relative to the DR detector 412.

Portions of the radiographic imaging system 460 described herein having remote controllable movement may each include a motor that is wirelessly controllable to rotate, extend/retract, or move along guides or tracks. Positioning of the tube head 601 allows accurate positioning of the x-ray source(s) therewithin in relation to the DR detector 412 and patient P. After controllably positioning the tube head 601 in relation to DR detector 412 and patient P, for example, the x-ray source therewithin may be remotely and controllably fired to emit an x-ray beam 606 to expose patient P and capture a radiographic image thereof in DR detector 412.

### ALTERNATIVE EMBODIMENTS

In one embodiment, a calibration module, as a program code portion of the data processing system 462, after one time calibration may be able to establish a coordinate transformation relation between the visualization module and the positioning module. Through error calibration, this embodiment is capable of enhancing the accuracy of the coordinate transformation between the visualization module and the positioning module, as well as the accuracy of the positioning module itself.

With the visualization module installed around or beside the x-ray source assembly 416, this embodiment is able to collect the video stream during the patient P alignment process. Combining the transformation information provided by the positioning module (transmitters/receivers), this embodiment will be able to display the boundary of the DR detector 412 and AEC sensors 442 position in real world and in real time, which makes the x-ray image acquisition process more intuitive, easy and unintimidating. This embodiment also allows users to choose between automatically aligning the radiation source assembly 416 and the DR detector 412, or manually aligning them using the guidance information described herein.

With an automatic alignment module, as a coded portion of the data processing system 462, this embodiment is capable of automatically controlling the x-ray source assembly 416 to align with the DR detector 412, based on the coordinate transformation relation provided by the positioning module. With the data processing module 462, all the related calibration, rendering and video stream processing are executed or calculated on it.

The positioning module enables the x-ray source assembly 416 to track the location of the DR detector 412, then a coordinate transformation relation between the x-ray source assembly 416 and DR detector 412 is established. The visualization module is utilized to collect the video stream for the following functionality modules. The calibration module provides the one-time calibration functionality to determine the coordinate transformation relation between the visualization module and the positioning module. Furthermore, an error calibration functionality is also provided to enhance the accuracy of coordinate transformation relation between the visualization module and the positioning module, as well as the accuracy of the positioning module itself. Next, a visualization information and positioning information fusion can be achieved through the data processing module. The guidance information is able to be rendered and drawn through the data processing module 462 and then displayed through the visualization module. With the guidance information displayed, such as the DR detector 412 boundary, the x-ray source assembly 416 can be manually aligned with the DR detector 412. Or the automatic alignment module provides the functionality to automatically control the x-ray source assembly 416 to align with the DR detector 412, based on the coordinate transformation relation provided by the positioning module.

In one embodiment, the positioning module consists of one or more signal emitters and signal receivers 413, 414. One or more signal emitters will be installed near or around the x-ray source assembly 416, while one or more signal receivers will be installed on the side of the DR detector 412 (FIG. 5). The signal emitters and receivers could be but not limited to a magnetic field based navigation system, an ultra-wide band based location system, or an inertial measurement unit based system.

In another embodiment, the positioning module can provide the coordinate transformation between the signal emitters and the signal receivers. And the physical installation positions of the signal emitters and signal receivers relative to the center of the x-ray source assembly 416 and DR detector 412 are known. Thus, the related coordinate transformation between the x-ray source assembly 416 and DR detector 412 is obtained accordingly.

In another embodiment, the visualization module consists of one or more video capable cameras 430 and one digital display 464. The camera could be but not limited to alive video camera, a 2D camera, or a RGB-Depth camera, which can be utilized to collect the video stream during the patient positioning. Then the visualization module can display either the original video stream or the video stream with guidance information through the digital display 464.

In another embodiment, the calibration module can provide two kinds of calibration functionality. A coordinate transformation between the signal emitter of the positioning module and the camera of the visualization module can be established through the one-time calibration functionality.

In another embodiment, the coordinate transformation between the camera of the visualization module and the DR detector 412 can be established by creating a world coordinate system on the DR detector 412 and then performing camera calibration using landmarks on the DR detector 412.

In another embodiment, the coordinate transformation between the signal receivers and the DR detector 412 can be determined by measuring the physical distance between them. Then a coordinate transformation between the camera of visualization module and the signal emitter is established accordingly.

In another embodiment, the error calibration is implemented to compensate the error induced by the installation and the environmental interference. This calibration module could consist of, but is not limited to, a pretrained deep learning network or a traditional machine learning model relying on engineered features. Pairs of data containing precise and biased transformation information between the visualization module and the positioning model, as well as between the signal emitter and the receiver, will be fed into an artificial intelligence network for training. Thus, the trained model is capable to compensate the error induced by the installation and environmental interference.

In another embodiment, the data processing module 462 will handle all the calibration, rendering and video stream processing. In one embodiment, the data processing module 462 could be but not limited to a personal computer, an embedded industrial control computer, or a cloud server. In another embodiment, the data processing module 462 can calculate the coordinate transformation relationship between the positioning module and the visualization module. Additionally, it can fuse and render the positioning and visualization information, drawing guidance information such as DR detector 412 boundaries and Automatic Exposure Control (AEC) boundaries onto the video stream. This allows the display of guidance information through the visualization module's digital display 464, providing guidance to the operator.

In another embodiment, the automatic alignment module provides automatic alignment functionality for the x-ray source assembly 416 and the DR detector 412. After obtaining the coordinate transformation information between the x-ray source assembly 416 and the DR detector 412 from the positioning module, the automatic alignment module can automatically align the x-ray source assembly 416 with the DR detector 412. This is achieved by controlling a mechanical device that adjusts the position of the x-ray source assembly 416 based on the coordinate transformation information, thereby completing the alignment process.

In another embodiment, the mechanical device that can fix the x-ray source assembly 416 and control its position can include, but is not limited to, a combination of motors with multiple degrees of freedom, an industrial robotic arm, or a medical robotic arm.

In another embodiment, operators can manually align the x-ray source assembly 416 and the DR detector 412 based on the guidance provided by the visualization module according to their actual needs and usage scenarios. Alternatively, they can choose the alignment function provided by the automatic alignment module to allow the system to automatically align the x-ray source assembly 416 and the DR detector 412.

In one example configuration, the visualization module can be installed on the left side of a x-ray source assembly 416, including one RGBD camera and one portable digital display 464. The visualization module is connected to a PC (data processing module 462) to start the video stream. One-time calibration and error calibration may be performed in sequence through the system calibration module to initialize the positioning module. Then the positioning and video information can be fused through the data processing module 462 to generate guidance information (Detector boundary and AEC boundary) which will be displayed on a portable monitor for the operator. The positioning information will also be transmitted to the automatic alignment module to initialize the alignment process.

In one workflow example, the patient and exam information can be input on PC side; the DR detector 412 is placed in the appropriate position; the x-ray source assembly 416 is moved to a position roughly above the DR detector 412; and the guidance information drawing function is activated to display the DR detector 412 boundary and AEC boundary on the video display. Operators can manually adjust the position and angle of the x-ray source assembly 416 based on the displayed guidance information to align it with the DR detector 412. Alternatively, operators can selectively activate the automatic alignment function, wherein the positioning device will automatically adjust the position and angle of the x-ray source assembly 416 to complete the alignment. After, the x-ray source assembly 416 is aligned with the DR detector 412, an exposure process can begin.

Accurately repeating alignment and exposure techniques in digital radiography imaging may be accomplished by the methods and apparatuses disclosed herein. In particular, imaging parameters may be recorded and thereafter duplicated in another imaging session to ensure that the same proper imaging methods are used.

Typically, for a fixed DR system, source-detector alignment is generally ensured by a complex and sophisticated mechanical device. Hence, a well-designed smart alignment system that offers both visual guidance information and automatic alignment between the x-ray source and the detector for out-of-bucky scenarios will not only significantly expedite the positioning process and enhance image quality, but also decrease the retake rate, leading to time and workload savings. The alignment techniques used for imaging as described herein may be measured and recorded, so that the recorded imaging settings may be retrieved in a subsequent imaging session and be reapplied for that session.

The present invention includes an embodiment to locate, track and record the position between the x-ray source and the DR detector 412. Subsequently, guide information may be retrieved and provided to an operator to assist in x-ray source positioning. Furthermore, the system can automatically align source and detector to ensure optimal alignment, thereby ensuring high-quality images and reducing the need for image retakes. These techniques can solve most of the problems associated with previous solutions, which were limited in their ability to apply to only in-bucky cases with limited support and guidance.

The present disclosure introduces an alignment system to overcome the problem and disadvantages of the previous solutions and methods. This system consists of a visualization module, a positioning module, a calibration module, an automatic alignment module and a data processing module 462. The location information of the x-ray source and the DR detector 412 is initially provided by the positioning module, which is then fused with a video stream captured from the visualization module through the data processing module 462. Those fused information will be drawn and displayed on the digital display 464 in real-time, so that it can provide a vivid guidance during the patient positioning process. Previously used and stored radiographic imaging parameters may be retrieved and displayed for use by an operator for setting up the imaging system in the same configuration as was used before. The system also includes a feature that can automatically aligns the x-ray source and the DR detector 412 according to the previously stored imaging parameters. aligning the x-ray source and the detector, or manually aligning them using the guidance information or the stored imaging parameter information mentioned above.

The guidance information is able to be rendered and drawn through the data processing module 462 and then displayed through the visualization module. With the guidance information and/or stored parameter information displayed, such as the SID, patient support angle, source-detector angle, energy levels, etc., the x-ray source can be manually aligned with the detector and the system parameters adjusted for patient imaging. Or the automatic alignment module provides the functionality to automatically control the x-ray source to align with the DR detector 412, based on the coordinate transformation relation provided by the positioning module.

In one workflow example, the patient and parameter information can be displayed to an operator; the detector is placed in the appropriate position; the x-ray source is moved to a position roughly above the detector; and the stored parameter information is used by the operator to repeat the imaging system set-up in the same way as previously recorded and used for the patient. Alternatively, an operator can selectively activate the automatic alignment/adjustment function, wherein the positioning device will automatically adjust the position and angle of the x-ray source to complete the alignment according to the stored parameter information. Then the imaging session can begin.

As will be appreciated by one skilled in the art, aspects of the present invention may be embodied as a system, method, computer algorithm, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Python, C++ or the like and conventional procedural programming languages, such as the “C” programming language or similar programming languages. The program code may execute entirely on the operator's computer (device), partly on the operator's computer, as a stand-alone software package, partly on the operator's computer and partly on a remote computer or entirely on the remote computer or server. In the latter scenario, the remote computer may be connected to the operator's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An imaging system for radiographically imaging a patient, the system comprising:
a manually movable x-ray source assembly, the x-ray source assembly including a transmitter and a video camera;
a free standing manually positionable digital radiographic (DR) detector including a receiver, wherein the transmitter and receiver are configured to communicate to determine a position of the x-ray source relative to the digital radiographic detector; and
a display screen coupled to the video camera to display a live video image of the patient, wherein the system is configured to display an outline of a current position of the DR detector overlayed on the video image of the patient while the x-ray source is being manually moved.

2. The system of claim 1, further comprising an automatic alignment device for automatically moving the x-ray source assembly to an imaging position aligned with the DR detector.

3. The system of claim 2, further comprising a motor driven movable tube head containing the x-ray source assembly for moving the tube head to the imaging position.

4. The system of claim 3, further comprising a motor for moving the imaging system over a floor to the imaging position.

5. A method of operating an x-ray imaging system comprising the steps of:
positioning a digital radiographic (DR) detector adjacent to a patient;
positioning a radiation source such that the patient is located between the DR detector and the radiation source;
displaying guidance information on a display screen, the guidance information for positioning the radiation source in a properly aligned imaging position;
positioning the radiation source according to the guidance information; and
capturing a radiographic image of the patient.

6. The method of claim 5, further comprising displaying a video image of the patient on the display screen together with the guidance information.

7. The method of claim 6, further comprising displaying on the display screen a cursor overlayed on the video image of the patient, the cursor indicating the locations of the borders of the DR detector.

8. The method of claim 7, further comprising activating an automatic alignment device for automatically moving the radiation source to the properly aligned imaging position.

9. An x-ray imaging system for capturing radiographic images of a patient, the system comprising:
an x-ray source including a transmitter;
a free standing manually positionable digital radiographic (DR) detector including a plurality of AEC sensors, the digital radiographic detector having a receiver, wherein the transmitter and receiver are configured to determine an orientation of the digital radiographic detector relative to the x-ray source, and wherein each of the plurality of AEC sensors are individually activatable; and
an AEC selection interface configured to receive an operator selection of one or more of the plurality of AEC sensors to be activated, wherein the x-ray imaging system is configured to activate the operator selected ones of the AEC sensors for radiographic imaging of the patient, and wherein the AEC selection interface indicates a current position of each of the plurality of AEC sensors relative to the detector based on the determined orientation of the digital radiographic detector.

10. The system of claim 9, further comprising an automatic alignment device for automatically moving the x-ray source to an imaging position aligned with the DR detector.

11. The system of claim 10, further comprising a motor driven movable tube head containing the x-ray source for moving the tube head to the imaging position.
